(19) United States
(12) Patent Application Publication
(10) Pub. No.: US 2024/0117314 A1
(43) Pub. Date: Apr. 11, 2024

(54) MODIFIED STEM CELL AND PREPARATION METHOD AND USE THEREOF

(71) Applicant: Gwo Xi Stem Cell Applied Technology Co., Ltd., Hsinchu County (TW)

(72) Inventors: Ruei-Yue Liang, Hsinchu County (TW); Chia-Hsin Lee, Hsinchu County (TW); Kai-Ling Zhang, Hsinchu County (TW); Po-Cheng Lin, Hsinchu County (TW); Ming-Hsi Chuang, Hsinchu County (TW); Yu-Chen Tsai, Hsinchu County (TW); Peggy Leh Jiunn Wong, Hsinchu County (TW)

(21) Appl. No.: 18/376,440

(22) Filed: Oct. 3, 2023

Related U.S. Application Data
(60) Provisional application No. 63/414,921, filed on Oct. 11, 2022.

(30) Foreign Application Priority Data
Sep. 19, 2023 (TW) 112135592

(51) Int. Cl. C12N 5/0775; A01N 1/02; A61K 35/28; A61P 3/10

(52) U.S. Cl. CPC C12N 5/0662; A01N 1/0284; A61K 35/28; A61P 3/10; C12N 2500/32; C12N 2500/38

(57) ABSTRACT

The present invention relates to a method for preparing a modified stem cell, including the following steps: a cell culture step: culturing stem cells in a first culture medium of a culture dish at a predetermined cell density, and removing the first culture medium after a first culture time to obtain a first cell intermediate; an activity stimulation step: preserving the first cell intermediate in a freezing container having a cell cryopreservation solution, and performing a constant temperature stimulation treatment or a variable temperature stimulation treatment for at least more than 1 day; and a product collection step: after completing the activity stimulation step, placing the freezing container in an environment at a thawing temperature for thawing, and then removing the cell cryopreservation solution to obtain the modified stem cell. The modified stem cell can release at least one or more of IL-4, IL-5, IL-13, G-CSF, Fractalkine, and EGF.

### CROSS-REFERENCE TO RELATED APPLICATION

The present application claims priority from U.S. Provisional Application No. 63/414,921 filed on Oct. 11, 2022 and Taiwanese Patent Application No. 112135592 filed on Sep. 19, 2023, the contents of which are incorporated herein by reference in their entirety.

### TECHNICAL FIELD

The present invention relates to the technical field of diabetes treatment, in particular to a modified stem cell capable of simultaneously releasing specific growth factors, cytokines and interleukins, and a culture method and use thereof.

### BACKGROUND

Diabetes mellitus is a metabolic disease in which blood glucose rises due to abnormal carbohydrate metabolism caused by insufficient secretion or dysfunction of insulin. According to the statistics from the International Diabetes Federation (IDF), the number of people with diabetes in the world reached 537 million in 2021, and the number of people with diabetes in Taiwan exceeded 2.2 million in 2020, which means that one in every ten people is diabetic, and the ratio is increasing year by year. Diabetes is also one of leading causes of complications such as heart disease, stroke, lower limb amputation, blindness and renal failure. Apparently, diabetes has imposed a serious health and economic burden on the global population.

In diabetic patients, pancreatic cells cannot produce enough insulin (insulin deficiency) or the insulin function deteriorates (insulin resistance), so that glucose cannot enter cells, resulting in the increase of blood glucose concentration and ultimately the development of diabetes. The current mainstream treatment of diabetes may only control the condition at most, and requires lifelong medication or insulin use, which can not effect a radical cure of diabetes. In addition, although there have been successful cases of pancreas transplantation in Taiwan, the process of organ transplantation is so complicated. People have to go through organ waiting and matching, but even then, it is not always possible to get a successful transplant. This makes it quite important to develop a convenient treatment that may be applied directly without waiting and matching.

At present, some studies have pointed out that certain specific growth factors, cytokines and interleukins, such as G-CSF, Fractalkine, EGF, IL-4, IL-5 and IL-13, may have a good therapeutic effect on diabetes and its complications. If a cell therapy product may secrete these active ingredients, it is possible to greatly improve therapeutic benefits of diabetes and its complications. According to experimental results, it is found that G-CSF may prevent the progression of diabetic nephropathy; and the administration of Fractalkine may promote islet cells to increase insulin secretion so as to improve glucose tolerance, reduce islet β cell apoptosis, and reduce neuroinflammation and thus diabetic retinopathy. In addition, EGF has also been found to increase insulin secretion and reduce blood glucose in diabetic mice in animal experiments. Three interleukins, IL-4, IL-5 and IL-13, have been shown to prevent the occurrence of insulitis and reduce the incidence of diabetes.

However, the prior art cannot allow the cell therapy product to produce the above 6 active substances simultaneously, and they can only be produced singly by chemical induction or gene transfer. Therefore, for the use of a cell therapy product in treatment of diabetes, it is crucial to prepare a cell therapy product capable of simultaneously producing the above 6 active substances in order to save the production cost.

### SUMMARY

In view of this, the present invention may simultaneously increase the production of 6 specific substances including growth factors, cytokines and interleukins by using a special temperature programming process, which is more efficient and safer than the prior art using small molecule induction or gene transfer and has novelty. In addition, the secretion of the 6 active substances in a cell therapy product treated by the process of the present invention is far better than that of an untreated control group, which is progressive. The present invention may be used for treating diabetes and its complications. Besides, the present invention ensures that produced cells may be cryopreserved and transported, which provides broad benefits for the future application and development of products and has industrial applicability.

That is, the present invention may provide a method for preparing a modified stem cell, including the following steps: a cell culture step: culturing stem cells in a first culture medium of a culture dish at a predetermined cell density, and removing the first culture medium after a first culture time to obtain a first cell intermediate; an activity stimulation step: preserving the first cell intermediate in a freezing container having a cell cryopreservation solution, and performing a constant temperature stimulation treatment or a variable temperature stimulation treatment for at least more than 1 day; and a product collection step: after completing the activity stimulation step, placing the freezing container in an environment at a thawing temperature for thawing, and then removing the cell cryopreservation solution to obtain the modified stem cell. The predetermined cell density is 6,000 to 15,000 cells/cm². The culture dish contains oxygen-containing functional groups, and thus features a net negative surface charge and hydrophilic on the surface thereof. The modified stem cell is capable of releasing at least one or more from a group consisting of G-CSF, Fractalkine, EGF, IL-4, IL-5 and IL-13.

In an embodiment of the present invention, the stem cell is any selected from an adipose-derived stem cell, a bone marrow stem cell, a peripheral blood stem cell and an umbilical cord blood stem cell. Preferably, the stem cell is any selected from an adipose-derived stem cell, a bone marrow stem cell and a peripheral blood stem cell. More preferably, the stem cell is selected from an adipose-derived stem cell or a bone marrow stem cell.

In an embodiment of the present invention, the culture dish contains at least more than 20% of oxygen-containing functional groups.

In an embodiment of the present invention, the constant temperature stimulation treatment is cryopreserving the first cell intermediate in a constant temperature environment within a temperature range of −100° C. to −200° C. for 1 to 1,825 days.

In an embodiment of the present invention, the variable temperature stimulation treatment includes: for the first cell intermediate, performing cryopreservation in an environment at a first freezing temperature for 1 to 1,825 days, subsequently performing cryopreservation in an environment at a second freezing temperature for 1 to 7 days, and then performing cryopreservation in an environment at a third freezing temperature for 1 to 365 days. The first freezing temperature, the second freezing temperature and the third freezing temperature are all below −50° C. The first freezing temperature is lower than the second freezing temperature, and the second freezing temperature is higher than the third freezing temperature.

In an embodiment of the present invention, the first freezing temperature and the third freezing temperature are respectively between −100° C. and −200° C.; the second freezing temperature is at least 30° C. higher than the first freezing temperature; and the second freezing temperature is at least 30° C. higher than the third freezing temperature.

In an embodiment of the present invention, the total time for the variable temperature stimulation treatment of the first cell intermediate cryopreserved at the first freezing temperature, the second freezing temperature and the third freezing temperature is at least more than 3 days.

In an embodiment of the present invention, the first culture medium is a Keratinocyte-SFM culture medium containing 1-100 mM of N-acetyl-L-cysteine (Sigma) and 0.05-50 mM of L-ascorbic acid 2-phosphate (Sigma).

In addition, the present invention may also provide a modified stem cell, prepared by performing cell culture and activity stimulation treatment on stem cells by the above method. The modified stem cell is capable of releasing at least one or more from a group consisting of G-CSF, Fractalkine, EGF, IL-4, IL-5 and IL-13, and may be used as a cell therapy product for treating diabetes and its complications.

In an embodiment of the present invention, the modified stem cell may be used for treating any of cardiovascular diseases, traumatic diseases, lung diseases, neurological diseases, immune diseases, liver diseases, endocrine diseases, skin diseases, gastrointestinal diseases, kidney diseases, hematological diseases, cancer and tumor diseases, gynecological diseases, mental diseases, urinary diseases, ophthalmic diseases and dental diseases, in addition to being used for treating diabetes.

### BRIEF DESCRIPTION OF THE DRAWINGS

FIG. 1A is a chart showing a comparison of the amounts of G-CSF released by stem cells S1, modified stem cells W1 and modified stem cells W2 in cytokine and growth factor content analysis.

FIG. 1B is a chart showing a comparison of the amounts of Fractalkine released by the stem cells S1, the modified stem cells W1 and the modified stem cells W2 in the cytokine and growth factor content analysis.

FIG. 1C is a chart showing a comparison of the amounts of EGF released by the stem cells S1, the modified stem cells W1 and the modified stem cells W2 in the cytokine and growth factor content analysis.

FIG. 1D is a chart showing a comparison of the amounts of IL-4 released by the stem cells S1, the modified stem cells W1 and the modified stem cells W2 in the cytokine and growth factor content analysis.

FIG. 1E is a chart showing a comparison of the amounts of IL-5 released by the stem cells S1, the modified stem cells W1 and the modified stem cells W2 in the cytokine and growth factor content analysis.

FIG. 1F is a chart showing a comparison of the amounts of IL-13 released by the stem cells S1, the modified stem cells W1 and the modified stem cells W2 in the cytokine and growth factor content analysis.

### DETAILED DESCRIPTION

For the implementation of the present invention, different specific embodiments will be listed below to provide more detailed description and illustration, in order to make the spirit and content of the present invention more complete and easy to understand. However, it should be appreciated by those of ordinary skill in the art that the present invention is definitely not limited to these examples, and the present invention may be achieved by using other identical or equivalent functions and step sequences.

All technical and scientific terms used herein have the same meaning as being commonly understood by those of ordinary skill in the art to which the present invention belongs. In addition, unless clearly conflicted in the context otherwise, a singular term used herein should include the plural, and a plural term should include the singular.

Unless otherwise defined herein, the term “treat/treating/treatment” means an action administered to a patient with a specific disease or disorder. The action may alleviate the patient's disease or disorder, or reduce the severity of one or more symptoms, or slow down or delay the progress of the disease or disorder.

Herein, the terms “individual” or “patient” may be used interchangeably. The term “individual” or “patient” refers to an animal that may be treated by compounds and/or methods respectively, including but not limited to for example dogs, cats, horses, sheep, pigs, cattle and the like, as well as humans and non-human primates. Unless otherwise specified, the “individual” or “patient” may include male and female. In addition, it also includes individuals or patients, preferably humans, suitable for being treated by the pharmaceutical composition and/or method of the present invention.

Although numerical ranges and parameters illustrating the broad scope of the present invention are approximations, numerical values set forth in specific examples are reported as accurately as possible. However, any numerical value inherently contains some errors inevitably caused by a standard deviation in its respective test measurement. Herein, the term “about” generally means that an actual value is within 10%, 5%, 1% or 0.5% above or below a specific value or range. Alternatively, the term “about” indicates that the actual value falls within an acceptable standard error of a mean when considered by those of ordinary skill in the art. Unless explicitly indicated in the examples or otherwise, all ranges, amounts, values and percentages used herein (for example, for describing amount of material, time, temperature, operating conditions, amount ratio and the like) should be understood as being modified by the word “about”. Therefore, unless explicitly stated to the contrary, all numerical parameters disclosed in this specification and the appended claims are approximations and may be changed if necessary. In any case, each numerical parameter should be interpreted at least in accordance with reported significant figures and by applying ordinary rounding techniques.

First, a method for preparing an optimized cell graft of the present invention will be described. The method includes the following steps:
a cell culture step: culturing stem cells in a first culture medium of a culture dish at a predetermined cell density, and removing the first culture medium after a first culture time to obtain a first cell intermediate;
a cell activity stimulation step: preserving the cell intermediate in a freezing container having a cell cryopreservation solution, and performing a constant temperature stimulation treatment or a variable temperature stimulation treatment for at least more than 1 day; and
a product collection step: after completing the activity stimulation step, placing the freezing container in an environment at a thawing temperature for thawing, and then removing the cell cryopreservation solution to obtain the modified stem cell.

The stem cell referred to in the present invention is a mesenchymal stem cell (MSC), which may be isolated from human tissues such as bone marrow, fat, teeth, peripheral blood, umbilical cord, umbilical cord blood, amniotic membrane, placenta, etc. In general, the stem cell is any selected from an adipose-derived stem cell, a bone marrow stem cell, a peripheral blood stem cell and an umbilical cord blood stem cell. Preferably, the stem cell is any selected from an adipose-derived stem cell, a bone marrow stem cell and a peripheral blood stem cell. More preferably, the stem cell is selected from an adipose-derived stem cell or a bone marrow stem cell.

The aforementioned cell culture step mainly involves expanding the number of the stem cells. The predetermined cell density of the stem cells before expansion is generally 6,000 to 15,000 cells/cm². The first culture medium is a Keratinocyte-SFM culture medium containing 1-100 mM of N-acetyl-L-cysteine (Sigma) and 0.05-50 mM of L-ascorbic acid 2-phosphate (Sigma). The culture dish containing oxygen-containing functional groups is negatively charged and hydrophilic, which has higher hydrophilicity and wettability as compared with a general standard TC (Tissue culture) treated culture dish and may promote cell attachment and spreading.

Next, the activity stimulation step mainly involves moving the cell intermediate into a low temperature environment and stimulating cell activity by the constant temperature stimulation treatment or the variable temperature stimulation treatment.

In an embodiment of the present invention, the constant temperature stimulation treatment is to move the cell intermediate into a constant temperature environment within a temperature range of −100° C. to −200° C. and perform cryopreservation for 1 to 1,825 days, preferably 1 to 1,460 days, more preferably 1 to 1,095 days, and most preferably 1 to 875 days.

Additionally, the temperature in the constant temperature environment generally ranges from −100° C. to −200° C., preferably from −140° C. to −200° C., more preferably from −180° C. to −200° C.

Also, in another embodiment of the present invention, the variable temperature stimulation treatment includes the following steps:
a first freezing step: for the cell intermediate, performing cryopreservation in an environment at a first freezing temperature for 1 to 1,825 days, preferably 1 to 1,460 days, more preferably 1 to 1,095 days, and most preferably 1 to 887 days;
a second freezing step: performing cryopreservation for the cell intermediate in an environment at a second freezing temperature for 1 to 7 days, preferably for 1 to 5 days, more preferably for 1 to 4 days, and most preferably for 1 to 3 days; and
a third freezing step: performing cryopreservation for the cell intermediate in an environment at a third freezing temperature for 1 to 365 days, preferably 1 to 180 days, more preferably 1 to 90 days, and most preferably 1 to 33 days.

According to the technical idea of the present invention, the first freezing temperature, the second freezing temperature and the third freezing temperature are all below −50° C., the first freezing temperature is lower than the second freezing temperature, and the second freezing temperature is higher than the third freezing temperature. In addition, the first freezing temperature and the third freezing temperature may be the same or different, and the first freezing temperature may be higher than, lower than or equal to the third freezing temperature, but the temperature difference between them is within 100° C. or below.

Also, the second freezing temperature is at least 30° C., preferably 50° C., more preferably 80° C., and most preferably 120° C. higher than the first freezing temperature. The second freezing temperature is at least 30° C., preferably 50° C., more preferably 80° C., and most preferably 120° C. higher than the third freezing temperature.

Furthermore, the first freezing temperature generally ranges from −100° C. to −200° C., preferably from −140° C. to −200° C., more preferably from −180° C. to −200° C.

Also, the second freezing temperature generally ranges from −60° C. to −90° C., preferably from −65° C. to −85° C., more preferably from −67° C. to −83° C.

In addition, the third freezing temperature generally ranges from −100° C. to −200° C., preferably from −140° C. to −200° C., more preferably from −180° C. to −200° C.

In addition, according to the technical idea of the present invention, the cell cryopreservation solution used in the embodiments is not particularly limited. Generally, the cell cryopreservation solution is prepared by adding 5 wt % to 15 wt % of Dimethyl sulfoxide (DMSO) into the first culture medium, or it may be a commercially available cell cryopreservation solution product containing 5 wt % to 15 wt % of DMSO.

The product collection step mainly involves thawing the cell after the activity stimulation step. The modified stem cell may be separated and removed from the cell cryopreservation solution by centrifugation. This step is usually carried out when the modified stem cell is ready for use in the treatment of a patient. The thawing temperature generally ranges from 30° C. to 40° C.

The modified stem cell obtained by the above method is capable of simultaneously releasing a cytokine (G-CSF), growth factors (Fractalkine and EGF) and interleukins (IL-4, IL-5 and IL-13), and may be used as a cell therapy product for treating diabetes and its complications.

In addition, the modified stem cell may be used for treating any of cardiovascular diseases, traumatic diseases, lung diseases, neurological diseases, immune diseases, liver diseases, endocrine diseases, skin diseases, gastrointestinal diseases, kidney diseases, hematological diseases, cancer and tumor diseases, gynecological diseases, mental diseases, urinary diseases, ophthalmic diseases and dental diseases, in addition to being used for treating diabetes.

In order to more fully and completely describe the present invention, an illustrative description will be given for the implementations and specific examples of the present invention. However, this is not intended to represent the only form that may be practiced or utilized in the specific examples of the present invention. The features and configurations of multiple specific examples as well as the process steps and sequence of operating these specific examples are encompassed in the embodiments. However, in other examples, this may also be done by the same or equivalent functions and sequence of steps.

### Comparative Example 1

About 5 g of adipose tissue was collected from the abdomen of a subject during abdominal surgery. This research was approved by the hospital ethics committee, and an informed consent from the subject was received. First, the adipose tissue was chopped into fragments smaller than 3 mm by scissors and a scalpel, and human stem cells were isolated and purified in the following steps.

The chopped adipose tissue was mixed homogeneously and gently with an equal volume of phosphate buffer solution (PBS), and then left for a short period of time so that the mixture was divided into two layers. The upper solution (containing stem cells, adipocytes, and blood) was washed twice with PBS, and then the tissue was dissociated with 0.075% collagenase (Type I) in an environment at 36.5-38.5° C. for 60 minutes. Afterwards, the dissociated tissue was mixed homogeneously with an equal volume of DMEM culture medium (GIBCO-BRL) containing 10% fetal bovine serum (FBS), and the resulting mixture was cultured at room temperature for 10 minutes. By then, the mixture was divided into two layers. The lower solution was centrifuged at 1,500 rpm at 20° C. for 5 minutes. The supernatant was removed, and the remaining bottom cells were broken up with 160 mM of NH₄Cl to eliminate red blood cells. The solution was filtered through a 40 mm sieve, and the filtrate was collected in a new tube. An equal volume of DMEM culture medium (GIBCO-BRL) containing 10% fetal bovine serum (FBS) was added to the solution, and homogeneous mixing was performed. The resulting mixture was washed by centrifugation at 1,500 rpm at 20° C. for 5 minutes. Afterwards, the supernatant was removed, and the bottom cells were primary stem cells.

The primary stem cells were cultured in a DMEM culture medium (GIBCO-BRL) containing 10% fetal bovine serum (FBS) in a TC (Tissue culture) treated culture dish.

After culturing to day 7, the stem cells were removed and placed in a cell cryopreservation solution with a DMSO concentration of 10 wt % for dispensing, with a cell count of 3×10\hat{ }7±20% cells/mL per cryotube. Then, the cryotubes were cryopreserved in an environment at −196±10° C.

After one month of cryopreservation, the cryotubes containing frozen cells were removed and immediately placed in an incubator at 37° C. for 3 minutes, followed by transferring the cryotubes into a biological safety cabinet (BSC) and wiping the outside of the cryotubes with 75% ethanol before opening them. 1 mL of cell suspension was transferred into a centrifuge tube containing 9 mL of normal saline and centrifuged at 300×g for 5 minutes.

Next, the supernatant was removed, and the cell pellets were gently placed in 10 mL of saline and resuspended therein. Then, the cell suspension was centrifuged at 300×g at 22° C. for 5 minutes, and the supernatant was removed. After repeating this step three times, the cell pellets were resuspended in 10 mL of saline to obtain stem cells S1.

### Embodiment 1

Liposuction was performed on healthy donors during abdominal surgery to collect 2-5 g of adipose tissue from subcutaneous fat of the abdominal wall. The liposuction procedure took approximately 1 hour or less, and the wound was smaller than 1 cm. All donors provided written consents. The human adipose tissue was placed in a phosphate buffer solution (PBS) without Ca²⁺/Mg²⁺ and immediately transferred to a laboratory.

The human adipose tissue was removed from a transport culture medium and placed in a culture dish. The adipose tissue was washed with a phosphate buffered saline (PBS) without Ca²⁺/Mg²⁺ 3 to 4 times, and chopped into small fragments (about 1-3 mm³ in volume). The tissue was dissociated with 0.1-0.3% collagenase in an environment at 36.5-38.5° C. for 60 minutes. After the digestion of the collagenase, cells and undigested tissue fragments were isolated from granules of stromal vascular fractions (SVF) by centrifugation at 20-25° C., 500 g for 5-15 minutes. The dissociated cells were collected and cultured in an incubator with a 5% CO₂ at 36.5-38.5° C. After 1-2 days of culture, the supernatant and fragments were removed from the culture to obtain primary stem cells.

The primary stem cells were subjected to expansion culture, and the culture medium and culture conditions being used were as follows:

0.5×10⁵ primary stem cells were placed in a culture dish made of a material containing more than 20% of oxygen-containing functional groups and cultured in a Keratinocyte-SFM culture medium (Gibco) containing 1-100 mM of N-acetyl-L-cysteine (Sigma) and 0.05-50 mM of L-ascorbic acid 2-phosphate (Sigma) for 7 days. The culture environment was a cell incubator with a temperature controlled at 36.5-38.5° C. and containing 5% carbon dioxide.

After culturing to day 7, the stem cells were removed and placed in a cell cryopreservation solution with a DMSO concentration of 10 wt % for dispensing, with a cell count of 3×10\hat{ }7±20% cells/mL per cryotube. Then, the cryotubes were cryopreserved in an environment at −196±10° C.

After one month of cryopreservation, the cryotubes containing frozen cells were removed and immediately placed in an incubator at 37° C. for 3 minutes, followed by transferring the cryotubes into a biological safety cabinet (BSC) and wiping the outside of the cryotubes with 75% ethanol before opening them. 1 mL of cell suspension was transferred into a centrifuge tube containing 9 mL of normal saline and centrifuged at 300×g for 5 minutes.

Next, the supernatant was removed, and the cell pellets were gently placed in 10 mL of saline and resuspended therein. Then, the cell suspension was centrifuged at 300×g at 22° C. for 5 minutes, and the supernatant was removed. After repeating this step three times, the cell pellets were resuspended in 10 mL of saline to obtain modified stem cells W1.

### Embodiment 2

Primary stem cells were obtained by using the same method as in embodiment 1. The primary stem cells were subjected to expansion culture, and the culture medium and culture conditions being used were as follows:

0.5×10⁵ primary stem cells were placed in a culture dish made of a material containing more than 20% of oxygen-containing functional groups and cultured in a Keratinocyte-SFM culture medium (Gibco) containing 1-100 mM of N-acetyl-L-cysteine (Sigma) and 0.05-50 mM of L-ascorbic acid 2-phosphate (Sigma) for 7 days. The culture environment was a cell incubator with a temperature controlled at 36.5-38.5° C. and containing 5% carbon dioxide.

After culturing to day 7, the stem cells were removed and placed in a cell cryopreservation solution with a DMSO concentration of 10 wt % for dispensing, with a cell count of 3×10\hat{ }7±20% cells/mL per cryotube. Then, the cryotubes were cryopreserved in an environment at −196±10° C. for 30 days, subsequently in an environment at −70±10° C. for 2 days, and finally in an environment at −196±10° C. for 7 days.

After completing the cryopreservation, the cryotubes containing frozen cells were removed and immediately placed in an incubator at 37° C. for 3 minutes, followed by transferring the cryotubes into a biological safety cabinet (BSC) and wiping the outside of the cryotubes with 75% ethanol before opening them. 1 mL of cell suspension was transferred into a centrifuge tube containing 9 mL of normal saline and centrifuged at 300×g for 5 minutes.

Next, the supernatant was removed, and the cell pellets were gently placed in 10 mL of saline and resuspended therein. Then, the cell suspension was centrifuged at 300×g at 22° C. for 5 minutes, and the supernatant was removed. After repeating this step three times, the cell pellets were resuspended in 10 mL of saline to obtain modified stem cells W2.

Cytokine and Growth Factor Content Analysis

The contents of cytokines and growth factors in the stem cells S1, the modified stem cells W1 and the modified stem cells W2 obtained in comparative example 1, embodiment 1 and embodiment 2 were analyzed.

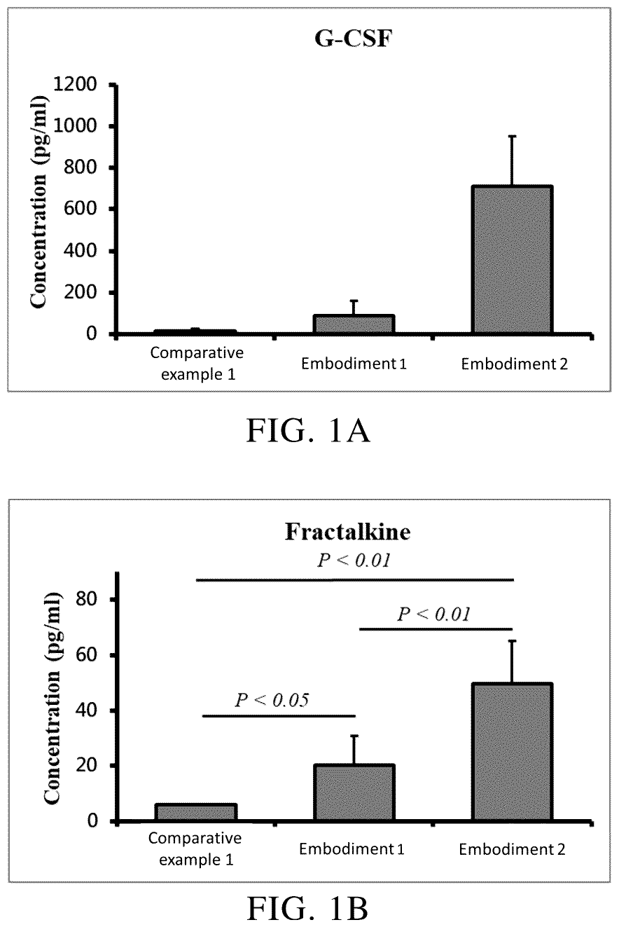

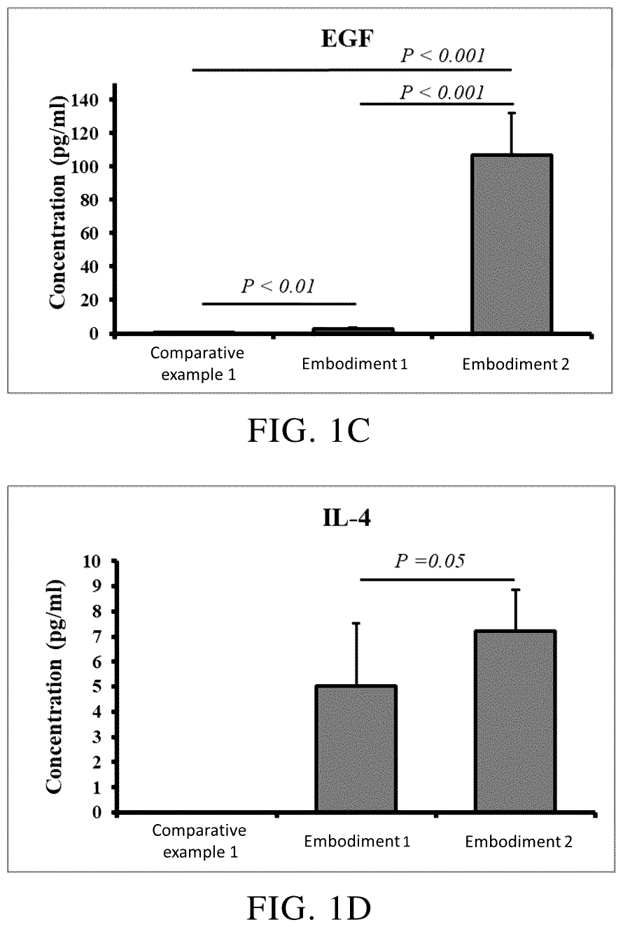

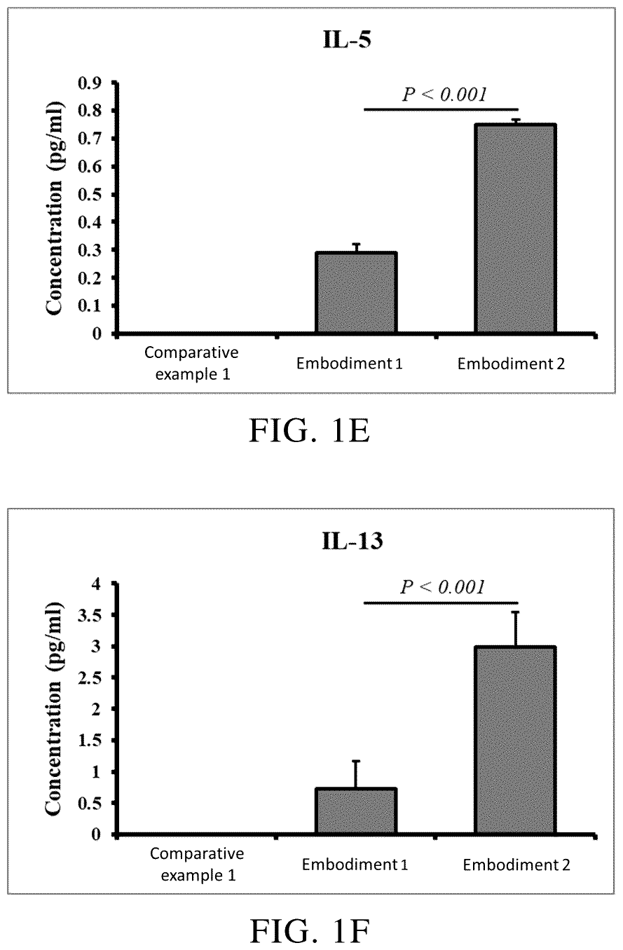

4×10⁵ cells were mixed with 0.5 mL of saline. The resulting mixture was allowed to stand in an environment at 2-10° C. for 24 hours and then centrifuged at 300×g for 5 minutes. The supernatant was collected and analyzed for the contents of G-CSF, Fractalkine, EGF, IL-4, IL-5 and IL-13 using MILLIPLEX® MAP MULIPLEX DETECTION (Merck Milliplex, instrument model: Luminex Magpix analyzer). Relevant values are recorded in Table 1 and plotted as FIG. 1A to FIG. 1F.

TABLE 1

| | Comparative | | | | | |
|---|---|---|---|---|---|---|
| | example 1 | | Embodiment 1 | | Embodiment 2 | |
| | (N = 3) | | (N = 6) | | (N = 6) | |
| Factor | Mean | SD | Mean | SD | Mean | SD |
| G-CSF(pg/ml) | 16.33 | 8.86 | 88.19 | 73.59 | 713.45 | 239.85 |
| Fractalkine(pg/ml) | 5.98 | 0.21 | 20.1 | 10.85 | 49.72 | 15.43 |
| EGF(pg/ml) | 0.57 | 0.16 | 2.71 | 0.98 | 106.89 | 25.39 |
| IL-4(pg/ml) | N.D. | 0 | 5.02 | 2.51 | 7.21 | 1.65 |
| IL-5(pg/ml) | N.D. | 0 | 0.29 | 0.03 | 0.75 | 0.02 |
| IL-13(pg/ml) | N.D. | 0 | 0.73 | 0.44 | 2.99 | 0.55 |

According to the results in Table 1 and FIG. 1A to FIG. 1F, it could be seen that the stem cells S1 obtained in comparative example 1 only release a small amount of G-CSF, Fractalkine and EGF, while the modified stem cells W1 and the modified stem cells W2 are capable of releasing a relatively large amount of G-CSF, Fractalkine and EGF. The amount of G-CSF released by the modified stem cells W1 is 5.4 times as large as that released by the stem cells S1, and the amount of G-CSF released by the modified stem cells W2 is 43.69 times as large as that released by the stem cells S1. The amount of Fractalkine released by the modified stem cells W1 is 3.36 times as large as that released by the stem cells S1, and the amount of Fractalkine released by the modified stem cells W2 is 8.31 times as large as that released by the stem cells S1. The amount of EGF released by the modified stem cells W1 is 4.75 times as large as that released by the stem cells S1, and the amount of EGF released by the modified stem cells W2 is 187.53 times as large as that released by the stem cells S1.

In addition, the stem cells S1 obtained in comparative example 1 do not release IL-4, IL-5 and IL-13, while the modified stem cells W1 and the modified stem cells W2 obtained in embodiments 1 and 2 are capable of releasing not only G-CSF, Fractalkine and EGF, but also IL-4, IL-5 and IL-13.

According to the above results, it could be seen that the method of the present invention may modify stem cells, so that the modified stem cells may secrete G-CSF, Fractalkine, EGF, IL-4, IL-5, and IL-13 at the same time, and has potential to serve as a cell therapy product for treating diabetes and its complications. The modified stem cells W2 contain higher concentrations of G-CSF, Fractalkine, EGF, IL-4, IL-5, and IL-13 compared to the modified stem cells W1.

### Embodiments 3 to 5

Primary stem cells were obtained by using the same method as in embodiment 1. The primary stem cells were subjected to expansion culture, and the culture medium and culture conditions being used were as follows:

0.5×10⁵ primary stem cells were placed in a culture dish made of a material containing more than 20% of oxygen-containing functional groups and cultured in a Keratinocyte-SFM culture medium (Gibco) containing 1-100 mM of N-acetyl-L-cysteine (Sigma) and 0.05-50 mM of L-ascorbic acid 2-phosphate (Sigma) for 7 days. The culture environment was a cell incubator with a temperature controlled at 36.5-38.5° C. and containing 5% carbon dioxide.

After culturing to day 7, the stem cells were removed and placed in a cell cryopreservation solution with a DMSO concentration of 10 wt % for dispensing, with a cell count of 3×10\hat{ }7±20% cells/mL per cryotube. Then, the cryotubes were cryopreserved in an environment at −196±10° C. for 14 days (embodiment 3), 875 days (embodiment 4) and 136 days (embodiment 5) respectively.

After the completion of the cryopreservation, the cryotubes containing frozen cells were removed and immediately placed in an incubator at 37° C. for 3 minutes, followed by transferring the cryotubes into a biological safety cabinet (BSC) and wiping the outside of the cryotubes with 75% ethanol before opening them. 1 mL of cell suspension was transferred into a centrifuge tube containing 9 mL of normal saline and centrifuged at 300×g for 5 minutes.

Next, the supernatant was removed, and the cell pellets were gently placed in 10 mL of saline and resuspended therein. Then, the cell suspension was centrifuged at 300×g at 22° C. for 5 minutes, and the supernatant was removed. After repeating this step three times, the cell pellets were resuspended in 10 mL of saline to obtain stem cells W3, W4 and W5 which were cryopreserved for different time respectively.

### Embodiments 6 to 8

Primary stem cells were obtained by using the same method as in embodiment 1. The primary stem cells were subjected to expansion culture, and the culture medium and culture conditions being used were as follows:

0.5×10⁵ primary stem cells were placed in a culture dish made of a material containing more than 20% of oxygen-containing functional groups and cultured in a Keratinocyte-SFM culture medium (Gibco) containing 1-100 mM of N-acetyl-L-cysteine (Sigma) and 0.05-50 mM of L-ascorbic acid 2-phosphate (Sigma) for 7 days. The culture environment was a cell incubator with a temperature controlled at 36.5-38.5° C. and containing 5% carbon dioxide.

After culturing to day 7, the stem cells were removed and placed in a cell cryopreservation solution with a DMSO concentration of 10 wt % for dispensing, with a cell count of 3×10\hat{ }7±20% cells/mL per cryotube. Then, the cryotubes were cryopreserved under the following conditions:

Embodiment 6: the cryotubes were first cryopreserved in an environment at −196±10° C. for 17 days, subsequently in an environment at −70±10° C. for 2 days, and finally in an environment at −196±10° C. for 33 days.

Embodiment 7: the cryotubes were first cryopreserved in an environment at −196±10° C. for 887 days, subsequently in an environment at −70±10° C. for 2 days, and finally in an environment at −196±10° C. for 7 days.

Embodiment 8: the cryotubes were first cryopreserved in an environment at −196±10° C. for 18 days, subsequently in an environment at −70±10° C. for 2 days, and finally in an environment at −196±10° C. for 3 days.

After completing the cryopreservation, the cryotubes containing frozen cells were removed and immediately placed in an incubator at 37° C. for 3 minutes, followed by transferring the cryotubes into a biological safety cabinet (BSC) and wiping the outside of the cryotubes with 75% ethanol before opening them. 1 mL of cell suspension was transferred into a centrifuge tube containing 9 mL of normal saline and centrifuged at 300×g for 5 minutes.

Next, the supernatant was removed, and the cell pellets were gently placed in 10 mL of saline and resuspended therein. Then, the cell suspension was centrifuged at 300×g at 22° C. for 5 minutes, and the supernatant was removed. After repeating this step three times, the cell pellets were resuspended in 10 mL of saline to obtain modified stem cells W6, W7 and W8 respectively.

Next, the modified stem cells W3 to W8 obtained in the above embodiments 3 to 8 were subjected to cell assays involving cell surface marker, cell viability, endotoxin, mycoplasma, sterility testing, and cytokine and growth factor content analysis.

Cell Assay-Cell Surface Marker

After thawing the modified stem cells W3 to W8, 100 μL of 1×10⁶/mL stem cells were put into a microcentrifuge tube, and fluorescently-labeled CD73, CD90, CD105, CD14, CD19, CD34, CD45 and HLA-DR (Becton Dickinson) antibodies were added at a ratio of 1:100. The stem cells and the antibodies were mixed homogeneously, and left to stand in the dark. Then, cell markers were analyzed using a BD AccuriC6 flow cytometer (Becton Dickinson). After the analysis was completed, the obtained results were recorded in the field of cell surface marker in Table 2.

Cell Assay-Cell Viability

After thawing the modified stem cells W3 to W8, cell viability was assessed by an ADAM-MC™ Automatic Cell counter (Digital Bio, NanoEnTek Inc.). After the analysis was completed, the obtained results were recorded in the field of cell viability in Table 2.

Cell Assay-Endotoxin

After thawing and washing the modified stem cells W3 to W8, the cell pellets were resuspended in 10 mL of saline. Then, the cell suspension was centrifuged at 300×g at 22° C. for 5 minutes. Afterwards, the supernatant was taken for endotoxin testing. This test was performed by a kinetic chromogenic assay based on LAL-Endotoxin reaction. In this reaction, a colorless artificial polypeptide substrate was added, and the substrate (pNA) would appear yellow when being cleaved by an enzyme. The absorbance value at OD405 nm was detected, thus quantifying the endotoxin content of standards and test samples having different concentrations in the reaction process. After the analysis was completed, the obtained results were recorded in the field of endotoxin in Table 2.

Cell Assay-Mycoplasma

After thawing and washing the modified stem cells W3 to W8, the cell pellets were resuspended in 10 mL of saline. Then, the cell suspension was centrifuged at 300×g at 22° C. for 5 minutes. Afterwards, the supernatant was taken for mycoplasma testing. This test was performed according to a nucleic acid amplification technology (NAT) test which uses a nucleic acid extraction technology for isolation of DNAs and a quantitative real-time polymerase chain reaction (PCR) technology for detection. The principle is that a specific primer having a fluorescent probe is used to copy a large number of specific gene fragments in a short time, nucleic acid signals are amplified, fluorescence values thereof are then detected by an optical system, and an analysis is finally performed on the basis of the counted number of PCR quantification cycles (Cq) and fluorescence intensity. Thus, whether a sample was contaminated by mycoplasma may be checked by this method. After the analysis was completed, the obtained results were recorded in the field of mycoplasma in Table 2.

Cell Assay-Sterility Testing

After thawing and washing the modified stem cells W3 to W8, the cell pellets were resuspended in 10 mL of saline. Then, the cell suspension was centrifuged at 300×g at 22° C. for 5 minutes. Afterwards, the supernatant was taken for sterility testing. In this test based on direct inoculation for sterility testing, test samples were respectively inoculated into a fluid thioglycolate medium I to culture anaerobic bacteria or aerobic bacteria and inoculated into a soybean-casein digest medium to culture trace amounts of aerobic bacteria or fungi, and then, the two culture media were respectively cultured at 30 to 35° C. and 20 to 25° C. After at least 14 days of observation, the culture media were checked for microbial growth. The obtained results were recorded in the field of sterility testing in Table 2.

Cell Assay-Cytokine and Growth Factor Content Analysis

After thawing and washing the modified stem cells W3 to W8, 4×10⁵ cells were mixed with 0.5 mL of saline. The resulting mixture was allowed to stand in an environment at 2-10° C. for 24 hours and then centrifuged at 300×g for 5 minutes. The supernatant was collected and analyzed for the contents of G-CSF, Fractalkine, EGF, IL-4, IL-5 and IL-13 using MILLIPLEX® MAP MULIPLEX DETECTION (Merck Milliplex, instrument model: Luminex Magpix analyzer). The obtained results were recorded in the field of cytokine and growth factor content analysis in Table 2.

TABLE 2

| | | | Embodiment | Embodiment | Embodiment |
|---|---|---|---|---|---|
| Embodiment | | | 3 | 4 | 5 |
| Modified stem cell | | | W3 | W4 | W5 |
| Constant | Time for constant | | 14 | 875 | 136 |
| temperature | temperature (−196 ± | | | | |
| stimulation | 10° C.) stimulation | | | | |
| treatment | treatment (days) | | | | |
| Variable | Time in environment | | N/A | N/A | N/A |
| temperature | at first temperature | | | | |
| stimulation | (−196 ± 10° C.) (days) | | | | |
| treatment | Time in environment | | N/A | N/A | N/A |
| | at second temperature | | | | |
| | (−70 ± 10° C.) (days) | | | | |
| | Time in environment | | N/A | N/A | N/A |
| | at third temperature | | | | |
| | (−196 ± 10° C.) (days) | | | | |
| Cell assay | Cell | CD73 | 98.78 | 99.39 | 95.91 |
| data | surface | CD90 | 99.95 | 100 | 99.96 |
| | marker | CD105 | 97.86 | 98.95 | 99.4 |
| | (%) | CD14 | ≤2 | ≤2 | ≤2 |
| | | CD19 | ≤2 | ≤2 | ≤2 |
| | | CD34 | ≤2 | ≤2 | ≤2 |
| | | CD45 | ≤2 | ≤2 | ≤2 |
| | | HLA-DR | ≤2 | ≤2 | ≤2 |
| | Cell viability (%) | | ≥70 | ≥70 | ≥70 |
| | Endotoxin (EU/mL) | | <0.05 | <0.05 | <0.05 |
| | Mycoplasma | | Negative | Negative | Negative |
| | Sterility testing | | Negative | Negative | Negative |
| | cytokine | G-CSF | 43.56 | 38.43 | 40.26 |
| | and | Fractalkine | 14.74 | 11.72 | 11.75 |
| | growth | EGF | 1.86 | 2.31 | 3.87 |
| | factor | IL-4 | 3.53 | 4.84 | 2.18 |
| | content | IL-5 | 0.26 | 0.28 | 0.27 |
| | analysis | IL-13 | 0.50 | 0.44 | 0.56 |
| | (pg/ml) | | | | |
| | | | Embodiment | Embodiment | Embodiment |
| Embodiment | | | 6 | 7 | 8 |
| Modified stem cell | | | W6 | W7 | W8 |
| Constant | Time for constant | | N/A | N/A | N/A |
| temperature | temperature (−196 ± | | | | |
| stimulation | 10° C.) stimulation | | | | |
| treatment | treatment (days) | | | | |
| Variable | Time in environment | | 17 | 887 | 18 |
| temperature | at first temperature | | | | |
| stimulation | (−196 ± 10° C.) (days) | | | | |
| treatment | Time in environment | | 2 | 2 | 2 |
| | at second temperature | | | | |
| | (−70 ± 10° C.) (days) | | | | |
| | Time in environment | | 33 | 7 | 3 |
| | at third temperature | | | | |
| | (−196 ± 10° C.) (days) | | | | |
| Cell assay | Cell | CD73 | 99.3 | 99.88 | 99.63 |
| data | surface | CD90 | 99.8 | 99.79 | 98.8 |
| | marker | CD105 | 95.6 | 95.48 | 95.23 |
| | (%) | CD14 | ≤2 | ≤2 | ≤2 |
| | | CD19 | ≤2 | ≤2 | ≤2 |
| | | CD34 | ≤2 | ≤2 | ≤2 |
| | | CD45 | ≤2 | ≤2 | ≤2 |
| | | HLA-DR | ≤2 | ≤2 | ≤2 |
| | Cell viability (%) | | ≥70 | ≥70 | ≥70 |
| | Endotoxin (EU/mL) | | <0.05 | <0.05 | <0.05 |
| | Mycoplasma | | Negative | Negative | Negative |
| | Sterility testing | | Negative | Negative | Negative |
| | cytokine | G-CSF | 989.65 | 763.78 | 764.4 |
| | and | Fractalkine | 69.78 | 25.66 | 44.22 |
| | growth | EGF | 118.30 | 132.25 | 74.48 |
| | factor | IL-4 | 6.54 | 10.58 | 6.53 |
| | content | IL-5 | 0.74 | 0.77 | 0.77 |
| | analysis | IL-13 | 4.00 | 2.39 | 2.73 |
| | (pg/ml) | | | | |

According to the results in Table 2 above, it could be seen that there is not too much difference among the cell assay data of the modified stem cells W3 to W5 subjected to the constant temperature stimulation treatment, no matter the modified stem cells were left in an environment at a constant temperature (−196±10¹C) for 14 days or even up to 875 days. The cell surface markers CD73, CD90 and CD105 are all kept above 95%, while CD14, CD19, CD34, CD45 and HLA-DR are less than or equal to 2%. Moreover, the cell viability is kept above 70%, the endotoxin is kept below 0.05 EU/mL, and the mycoplasma and the sterility testing are all negative. The results of the cytokine and growth factor content analysis show that G-CSF, Fractalkine, EGF, IL-4, IL-5 and IL-13 could be secreted stably, which indicates that the modified stem cells manufactured by the constant temperature stimulation treatment are stable in quality.

In addition, there is not too much difference among the cell assay data of the modified stem cells W6 to W8 subjected to the variable temperature stimulation treatment, no matter the modified stem cells were left in an environment at a first freezing temperature (−196±10° C.) for 17 days or even up to 887 days, in an environment at a second freezing temperature (−70±10° C.) for 2 days, or in an environment at a third freezing temperature (−196±10° C.) for 3 days or even up to 33 days. The cell surface markers CD73, CD90 and CD105 are all kept above 95%, while CD14, CD19, CD34, CD45 and HLA-DR are less than or equal to 2%. Moreover, the cell viability is kept above 70%, the endotoxin is kept below 0.05 EU/mL, and the mycoplasma and the sterility testing are all negative. The cytokine and growth factor content analysis shows that G-CSF, Fractalkine, EGF, IL-4, IL-5 and IL-13 could be secreted stably, which indicates that the modified stem cells manufactured by the variable temperature stimulation treatment are stable in quality.

From the above results, it could be seen that the cell assay data of the modified stem cells W3 to W8 are stable, which indicates that the modified stem cells have excellent quality, no matter they are manufactured by the constant temperature stimulation treatment or the variable temperature stimulation treatment. More importantly, as could be seen from the results of the cytokine and growth factor content analysis, the modified stem cells W6 to W8 manufactured by the variable temperature stimulation treatment (embodiments 6 to 8) are more capable of secreting higher concentrations of G-CSF, Fractalkine, EGF, IL-4, IL-5 and IL-13 than those manufactured by the constant temperature stimulation treatment (embodiments 3 to 5), demonstrating that the modified stem cells manufactured by the variable temperature stimulation treatment have greater potential to serve as a cell therapy product for treating diabetes and its complications.

### Application Example

In this application example, the modified stem cells W2 obtained in the above embodiment 2 were used for the clinical treatment of diabetic patients. This application example is carried out according to the ethical principles of the Declaration of Helsinki as well as local laws and regulations, following the current operational guidelines for good clinical trials for pharmaceuticals.

The trial process is described as follows.

A. 2 Weeks Prior to the Start of the Trial (Week −2)-Criteria Preparatory Stage

Subjects must meet the following conditions to be included in the criteria preparatory stage 2 weeks prior to the start of the trial:
(1) The patient was diagnosed with type 1 diabetes (T1D).
(2) The patient was over 5 years old.
(3) The patient was within 12 months after being diagnosed with type 1 diabetes (T1D) during the screening visit period.
(4) The blood test results at the time of onset met the following:
1. Fasting blood glucose ≥7 mmol/L.
2. Glycosylated hemoglobin (HbA1C) ≥6.5%.
3. Presence of at least one antibody related to T1D, such as ICA, GAD, ZnT8 or IAA.
(5) The patient was using insulin for blood glucose control at the time of screening the subjects for participation in the study.
(6) The patient had no other serious acute diseases requiring treatment.
(7) The patient agreed to use stem cell transplantation for treatment after the doctor had explained the benefits, risks and possible risks of the stem cell transplantation.
(8) The patient's parent (father/mother or legal guardian) was able read, write and understand the ICF form and agreed to sign a consent form for participation in the study.

Exclusion Conditions
(1) The patient had relevant evidence of renal insufficiency: for male, creatinine concentration in blood >1.5 mg/dl (or >133 mmol/L); and for female, creatinine concentration in blood >1.4 mg/dl (or >124 mmol/L).
(2) The patient was in a state of renal failure. Within the range of nephrotic syndromes, urine protein level >3.5 g/day or urine protein/creatinine ratio >2.7.
(3) The patient was seriously infected or infected with a hepatitis B virus, a hepatitis C virus, an HIV virus or tuberculosis.
(4) The patient had cardiovascular diseases, respiratory diseases (lung, fibrosis, chronic respiratory failure), liver diseases, cancer or neurological diseases.
(5) The patient had blood coagulation disorders (INR>1.5, PTT>40, PT>15).
(6) The patient took any anticoagulant.
(7) The patient took systemic steroids.
(8) The patient participated in another clinical study involving experimental drugs and/or medical equipment.
(9) The patient had a history of allergic reaction to anesthetics and/or antibiotics.

B. Trial Start-Up Stage (Week 0)

Subjects remained on normal medication continuously during week −2. At week 0, stem cells were modified by the method of embodiment 2 to obtain modified stem cells W2. Then, the modified stem cells were infused intravenously at a dosage of 1×10⁶ cells/Kg of patient's body weight over approximately 30 minutes.

After completing the infusion, the insulin consumption of the subjects prior to the infusion and 6 months after the infusion was recorded respectively. The C-Peptide and fasting blood glucose of the subjects were measured 6 months after the infusion, and the measurements were compared with the values prior to the infusion. The results are shown in Table 3.

TABLE 3

| | | After | |
|---|---|---|---|
| | Prior to | treatment | |
| Test items | treatment | (6 months) | Change |
| C-Peptide (ng/mL) | 0.16 | 0.94 | Increased by 0.78 |
| Fasting blood glucose | 19 | 4.5 | Reduced by 14.5 |
| (mmol/L) | | | |
| Insulin consumption (IU/day) | 35 | 24 | Reduced by 11 |

First, reference will be made to the value of C-peptide. C-peptide is an inactive peptide cut out by pancreatic β-cells while producing insulin from proinsulin, which represents an endogenous islet production, and the normal range of its value is 0.81-3.85 ng/mL. The value of this parameter for patients with type 1 diabetes is usually very low, and will be increased if insulin secretion in the pancreas recovers after treatment. Therefore, as could be seen from the results in Table 3, the modified stem cells W2 obtained by the method of the present invention, as a cell therapy product, are capable of effectively recovering insulin secretion.

Next, the value of fasting blood glucose will be mentioned. In general, the normal range of fasting blood glucose is 4-7 mmol/L, and a value being more than 7 mmol/L represents a risk of diabetes. In this application example, the fasting blood glucose of the patients was reduced from 19 mmol/L to 4.5 mmol/L 6 months after the infusion of the modified stem cells W2. Therefore, as could be seen from the results in Table 3, the modified stem cells W2 obtained by the method of the present invention, as a cell therapy product, are capable of reducing blood glucose.

In addition, the daily insulin consumption of the patients will be outlined. Patients with type 1 diabetes usually cannot produce insulin by themselves, and need to control blood glucose by injecting exogenous insulin every day. Under normal conditions, no insulin injection is required. The daily insulin consumption of the patients was reduced by 11 IU 6 months after the infusion of the modified stem cells W2, showing that the modified stem cells W2 obtained by the method of the present invention, as a cell therapy product, are capable of effectively reducing the injection amount of exogenous insulin.

To sum up, the content of the present invention has been illustrated by the above embodiments, but the present invention is not limited to these embodiments. Those of ordinary skill in the technical field to which the present invention belongs could make various changes and modifications without departing from the spirit and scope of the present invention. For example, if the technical contents exemplified in the foregoing embodiments are combined or altered to become new embodiments, these embodiments should be also regarded as part of the content of the present invention. Therefore, the scope claimed by the present invention also encompasses the appended claims and the scope defined thereby.

What is claimed is:

1. A method for preparing a modified stem cell, comprising the following steps:
a cell culture step: culturing stem cells in a first culture medium of a culture dish at a predetermined cell density, and removing the first culture medium after a first culture time to obtain a first cell intermediate;
an activity stimulation step: preserving the first cell intermediate in a freezing container having a cell cryopreservation solution, and performing a constant temperature stimulation treatment or a variable temperature stimulation treatment for at least more than 1 day; and
a product collection step: after completing the activity stimulation step, placing the freezing container in an environment at a thawing temperature for thawing, and then removing the cell cryopreservation solution to obtain the modified stem cell, wherein
the predetermined cell density is 6,000 to 15,000 cells/cm²;
the culture dish contains oxygen-containing functional groups, and thus features a net negative surface charge and hydrophilic on the surface thereof;
the thawing temperature is between 30° C. and 40° C.; and
the modified stem cell is capable of releasing at least one or more from a group consisting of G-CSF, Fractalkine, EGF, IL-4, IL-5 and IL-13.

2. The method for preparing a modified stem cell according to claim 1, wherein the culture dish contains at least more than 20% of oxygen-containing functional groups.

3. The method for preparing a modified stem cell according to claim 1, wherein the constant temperature stimulation treatment is cryopreserving the first cell intermediate in a constant temperature environment within a temperature range of −100° C. to −200° C. for 1 to 1,825 days.

4. The method for preparing a modified stem cell according to claim 1, wherein the variable temperature stimulation treatment comprises: for the first cell intermediate, performing cryopreservation in an environment at a first freezing temperature for 1 to 1,825 days, subsequently performing cryopreservation in an environment at a second freezing temperature for 1 to 7 days, and then performing cryopreservation in an environment at a third freezing temperature for 1 to 365 days, wherein
the first freezing temperature, the second freezing temperature and the third freezing temperature are all below −50° C., the first freezing temperature is lower than the second freezing temperature, and the second freezing temperature is higher than the third freezing temperature.

5. The method for preparing a modified stem cell according to claim 4, wherein the first freezing temperature and the third freezing temperature are respectively between −100° C. and −200° C.;
the second freezing temperature is at least 30° C. higher than the first freezing temperature; and
the second freezing temperature is at least 30° C. higher than the third freezing temperature.

6. The method for preparing a modified stem cell according to claim 4, wherein the total time for the variable temperature stimulation treatment of the first cell intermediate cryopreserved at the first freezing temperature, the second freezing temperature and the third freezing temperature is at least more than 3 days.

7. The method for preparing a modified stem cell according to claim 1, wherein the first culture medium is a Keratinocyte-SFM culture medium containing 1-100 mM of N-acetyl-L-cysteine and 0.05-50 mM of L-ascorbic acid 2-phosphate.

8. A modified stem cell, which is obtained by performing cell culture and activity stimulation treatment on stem cells using the method according to claim 1, and the modified stem cell is capable of releasing at least one or more from a group consisting of G-CSF, Fractalkine, EGF, IL-4, IL-5 and IL-13.

9. A use of a modified stem cell for preparing a drug for treating diabetes, wherein the modified stem cell is prepared by performing cell culture and activity stimulation treatment on stem cells by the method according to claim 1, and the modified stem cell is capable of releasing at least one or more from a group consisting of G-CSF, Fractalkine, EGF, IL-4, IL-5 and IL-13.

10. A cell therapy product for treating diabetes, which at least comprises the modified stem cell prepared by the method according to claim 1, and the modified stem cell is capable of releasing at least one or more from a group consisting of G-CSF, Fractalkine, EGF, IL-4, IL-5 and IL-13.

11. A cell therapy product for treating diabetes, which at least comprises the modified stem cell according to claim 8, and the modified stem cell is capable of releasing at least one or more from a group consisting of G-CSF, Fractalkine, EGF, IL-4, IL-5 and IL-13.
